(19) United States
(12) Patent Application Publication
(10) Pub. No.: US 2007/0293815 A1
(43) Pub. Date: Dec. 20, 2007

(54) Microprojection Array Application with Sculptured Microprojections for High Drug Loading

(75) Inventors: Keith Chan, Brookline, MA (US); Rajan Patel, Menlo Park, CA (US); Peter E. Daddona, Menlo Park, CA (US); Cedric Wright, Mountain View, CA (US); Neha Agarwal, Los Altos Hills, CA (US)

Correspondence Address: WILSON SONSINI GOODRICH & ROSATI & MACROFLUX CORP., 650 PAGE MILL ROAD, PALO ALTO, CA, 94304, US

(73) Assignee: ALZA CORPORATION, Mountain View, CA (US)

(21) Appl. No.: 11/740,205

(22) Filed: Apr. 25, 2007

Related U.S. Application Data
(60) Provisional application No. 60/795,009, filed on Apr. 25, 2006.

(51) Int. Cl. A61M 37/00

(52) U.S. Cl. 604/46

(57) ABSTRACT

A transdermal drug delivery system with microprojections for disrupting a body surface to an individual. At least some of the microprojections have a depression for increasing drug loading by a drug coating.

### CROSS-REFERENCE

This application claims the benefit of U.S. Provisional Application No. 60/795,009, filed Apr. 25, 2006, which application is incorporated herein by reference.

### BACKGROUND OF THE INVENTION

In the past, drug delivery has been mainly done though oral ingestion or by injection. Delivery through the skin seems an attractive alternative. However, the natural barrier function of the body surface, such as skin, presents a challenge to delivery therapeutics into circulation. Transdermal devices for the delivery of biologically active agents or drugs have been developed for maintaining health and therapeutically treating a wide variety of ailments. For example, analgesics, steroids, etc., have been delivered with such devices. Transdermal drug delivery can generally be considered to belong to one of two groups: transport by a “passive” mechanism or by an “active” transport mechanism. In the former, such as drug delivery skin patches, the drug is incorporated in a solid matrix, a reservoir, and/or an adhesive system.

There are various ways to increase transdermal delivery rates. One way to increase the transdermal delivery of agents is to pretreat the skin with, or co-deliver with the beneficial agent, a skin permeation enhancer. A permeation enhancer substance, when applied to a body surface through which the agent is delivered, enhances the transdermal flux of the agent such as by increasing the permselectivity and/or permeability of the body surface, and/or reducing the degradation of the agent.

Another type of transdermal drug delivery is active transport in which the drug flux is driven by various forms of energy. Iontophoresis, for example, is an “active” electrotransport delivery technique that transports solubilized drugs across the skin by an electrical current. The feasibility of this mechanism is constrained by the solubility, diffusion and stability of the drugs, as well as electrochemistry in the device. The transport of the agent is induced or enhanced by the application of an applied electrical potential, which results in the application of electric current, to deliver or enhance delivery of the agent.

However, at the present many drugs and pharmaceutical agents still cannot be efficiently delivered by conventional passive patches or electrotransport systems through intact body surfaces. There is an interest in the percutaneous or transdermal delivery of larger molecules such as peptides and proteins to the human body as increasing number of medically useful peptides and proteins become available in large quantities and pure form. The transdermal delivery of larger molecules such as peptides and proteins still faces significant challenges. In many instances, the rate of delivery or flux of large molecules such as polypeptides through the skin is insufficient to produce a desired therapeutic effect due to their large size and molecular weight. In addition, polypeptides, proteins, and many biologics are easily degraded during and after penetration into the skin, prior to reaching target cells. On the other hand, the passive transdermal flux of many low molecular weight compounds is too limited to be therapeutically effective.

Yet another method to increase transdermal flux (e.g., across skin) is to mechanically penetrate or disrupt the skin. This technique has been mentioned in, for example, U.S. Pat. No. 5,879,326 issued to Godshall et al., U.S. Pat. No. 3,814,097 issued to Ganderton et al., U.S. Pat. No. 5,279,544 issued to Gross et al., U.S. Pat. No. 5,250,023 issued to Lee et al., U.S. Pat. No. 3,964,482 issued to Gerstel et al., Reissue 25,637 issued to Kravitz et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365. These devices use piercing elements or microprojections of various shapes and sizes to pierce the outermost layer (i.e., the stratum corneum) of the skin. The microprojections disclosed in these references generally extend perpendicularly as an array from a thin, flat member, such as a pad or sheet. The microprojections in some of these devices are extremely small, some having dimensions (i.e., a microblade length and width) of only about 25-400μ and a microblade thickness of only about 5-50 μl. Other penetrating elements are hollow needles having diameters of about 10μ or less and lengths of about 50-100μ. These tiny stratum corneum piercing/cutting elements are meant to make correspondingly small microslits/microcuts in the stratum corneum for enhanced transdermal agent delivery or transdermal body analyte sampling therethrough. The perforated skin provides improved flux for sustained agent delivery or sampling through the skin. In many instances, the microslits/microcuts in the stratum corneum have a length of less than 150μ and a width that is substantially smaller than their length.

When microprojection arrays are used to improve delivery or sampling of agents through the skin, consistent, complete, and repeatable microprojection penetration is desired. Microprojection arrays generally have the form of a thin, flat pad or sheet with a plurality of microprojections extending roughly perpendicularly upward and are difficult to handle if they are too big. When an individual manually pushes the microprotrusion array on the skin by hand, the push force may be hard to control and may be uneven across the area of the array. Thus, mechanically actuated devices have been invented to apply a microprojection array to the stratum to effect microprojection skin piercing penetration in a more consistent and repeatable manner. However, even with the help of a mechanical actuator, a large microprojection array is still hard to apply to the body surface since body surfaces are generally not actually flat. Further, large microprojection arrays are inconvenient and uncomfortable for the patient. Because many chemical drugs are not highly potent, to deliver an effective amount of the drug, increasing the drug loading per unit planar area of a microprojection member holding the microprojection array is desirable. The ability to increase drug loading on the device can be critical for patient compliance and the successful application of such a device.

What is needed is a microprojection array that has increased capacity to hold drug compared to prior devices. The present invention provides system and methods of making and using such systems in which the microprojection array has sculptured microprojections for increasing surface area for loading one or more drugs.

### SUMMARY OF THE INVENTION

This invention is related to microprojection systems and methodology that provide a microprojection array for application of the microprojections to the stratum corneum. The microprojection array includes a plurality of microprojections that penetrate the stratum corneum to improve transport of an agent across the stratum corneum. At least some of the microprojections have a surface with a depression on the surface. A drug coating is coated on at least a portion of the microprojection covering the depression.

In accordance with another aspect of the invention, is a device for drug delivery including a microprojection array with a plurality of stratum corneum piercing microprojections for piercing stratum corneum, at least some of the microprojections having an elongated depression on the surface of the microprojection. A drug coating is coated on at least a portion of the microprojection covering the depression.

In a further aspect of the invention, in a device for drug delivery including a microprojection array with a plurality of stratum corneum piercing microprojections for piercing the stratum corneum, at least some of the microprojections having depressions are blade microprojections with a sharp cutting point.

In a further aspect of the invention, in a device for drug delivery including a microprojection array with a plurality of stratum corneum piercing microprojections for piercing stratum corneum, the microprojections having depressions have a depression located on one side of the microprojection. In a further embodiment, the microprojections have a depression located on two sides of the microprojection.

In another aspect of the invention, a device for drug delivery has a microprojection array having microprojections for piercing the stratum corneum to facilitate drug delivery wherein the microprojections have shafts and at least some of the depressions are elongated along at least a portion of the shaft. In a further aspect, a microprojection with an elongated shaft can have a curved surface bowing oppositely from the depression.

In a further aspect of the invention, a device for drug delivery has a microprojection array with a plurality of stratum corneum piercing microprojections for piercing stratum corneum and at least some of the microprojections have a throughhole for increasing the capacity to hold a drug coating.

In a further aspect of the invention, a device for drug delivery has a microprojection array with a plurality of stratum corneum piercing microprojections for piercing stratum corneum and at least some of the microprojections have an arrowhead tip or a tombstone tip. In a further embodiment of the invention, the microprojection array can have some microprojections have an arrowhead tip or a tombstone tip and some microprojections without either an arrowhead tip or a tombstone tip.

In accordance with another aspect of the invention, a device for drug delivery has a microprojection array having microprojections for piercing the stratum corneum to facilitate drug delivery wherein at least some of the microprojections have a portion that is thumbnail shaped having a surface with an elongated channel depression thereon. A drug coating coats at least a portion of the microprojection covering the elongated channel depression, or is disposed on the depression.

In another aspect, a device for drug delivery is provided in which a microprojection array has at least some microprojections having a surface with a depression thereon, at least some of the microprojections forming groups in which at least one of the microprojections has a depression and the group has a continuous drug coating that coats the microprojections to increase drug loading.

In another aspect, a device for drug delivery is provided in which a microprojection array has at least some microprojections having a surface with a depression thereon, at least some of the microprojections forming groups wherein at least some of the microprojections are grouped together in pairs where at least one microprojection projects at an angle to lean toward the other microprojection in the pair. In an alternative embodiment, the microprojections in the pair are substantially parallel to each other.

In another aspect, a device for drug delivery is provided in which a microprojection array has at least some microprojections having a surface with a depression thereon, at least some of the microprojections forming groups wherein at least some of the microprojections are grouped together in pairs and wherein each microprojection has a base. Further, the bases of the pair of microprojections can be spaced apart by less than 200 μm. Alternatively the bases of the microprojections can be spaced apart by 10 μm to 100 μm.

In another aspect, the present invention further provides a method of making a device with microprojections for piercing stratum corneum to facilitate drug delivery by forming on at least some of the microprojections a depression on the surface of a microprojection and coating a drug coating on at least a portion of the microprojection to cover the depression. In some embodiments where the microprojections are grouped together in pairs, the drug coating can coat the pair as a continuous coating to facilitate drug delivery. Alternatively, the drug coating can coat the pair of microprojections as a continuous coating near the tips of the microprojection. In another embodiment, only one of the microprojections in the pair of microprojections can have a depression and be coated with a drug. Alternatively, each microprojection can have a depression and be coated with a drug. Various shapes and configurations, materials of construction and drug coating parameters can be selected to result in the desired microprojection drug delivery device.

In another aspect, the present invention provides for a method for piercing the stratum-corneum for drug delivery. In another aspect is a method for forming a stratum-corneum piercing drug delivery apparatus with microprojections in groups or not in groups.

The inclusion of one or more depressions on the face of a microprojection in the device with stratum corneum piercing microprojections increases the surface area with similar volume of microprojection material. The increase in area due to the presence of the depressions occurs, preferably, mainly in the portions of the microprojections that extend out of the plane of the microprojection member. This increase in surface area thus can increase the capacity of the microprojection to capture drug coating material on the microprojection without requiring additional planar area, whereas otherwise a larger device with a larger volume and larger planar surface area would be required. The advantage provided by increased surface area without increasing volume and planar area is especially important for drugs that are less potent. Because large devices for piercing the stratum corneum are hard to handle and increase discomfort to the patient, the ability to increase drug loading on a device can be critical for patient compliance and the successful application of such a device. Thus, the present invention provides substantial benefits for drug delivery not available in the past.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a sectional view of an applicator device and microprojection array system according to the present invention.

FIG. 2 illustrates an isometric view in portion of a microprojection array system according to the present invention.

FIG. 3 illustrates an isometric view in portion of a microprojection embodiment with depression according to the present invention.

FIG. 4 illustrates an isometric view in portion of another embodiment of a microprojection having a different shape according to the present invention.

FIG. 5 illustrates an isometric view in portion of yet another embodiment of a microprojection having a different shape according to the present invention.

FIG. 6 illustrates an isometric view in portion of another embodiment of a microprojection having a throughhole according to the present invention.

FIG. 7 illustrates an isometric view in portion of another embodiment of a microprojection having a channel according to the present invention.

FIG. 8 illustrates an isometric view in portion of another embodiment of a microprojection having a thumbnail shape according to the present invention.

FIG. 9 illustrates an isometric view in portion of an embodiment of a group of microprojections according to the present invention.

FIG. 10 illustrates an isometric view in portion of an embodiment of a group of microprojections forming a pinnacle according to the present invention.

FIG. 11 illustrates a sectional side view in portion of another embodiment of a group of microprojections forming a pinnacle according to the present invention.

FIG. 12 illustrates a sectional side view in portion of yet another embodiment of a group of microprojections forming a pinnacle according to the present invention.

FIG. 13 illustrates a sectional side view in portion of yet another embodiment of a group of microprojections forming a pinnacle according to the present invention.

FIG. 14 illustrates an isometric view in portion of another embodiment of a microprojection having a tunnel, formed from two microblades according to the present invention.

FIG. 15 is a scanning electronmigraph showing a portion of an embodiment of a microprojection array that resulted from stacking two microblade arrays according to the present invention.

FIG. 16 is a scanning electronmigraph showing a portion of another embodiment of a microprojection array that resulted from stacking two microblade arrays according to the present invention, showing drug coating.

FIG. 17 is a scanning electronmigraph showing a portion of yet another embodiment of a microprojection array that resulted from stacking two microblade arrays according to the present invention, showing drug coating.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention relates to methods and devices for transdermal delivery of drugs with a microprojection array that has sculptured microprojections to increase the surface area for holding drug or biologically active agent. For example, the microprojection can be sculptured to have a depression, thus increasing the surface area available for loading a drug.

In describing the present invention, the following terms will be employed, and are defined as indicated below. As used in this specification and the appended claims, the singular forms “a,” “an” and “the” include plural references unless the content clearly dictates otherwise.

As used herein, the term “transdermal” refers to the use of skin, mucosa, and/or other body surfaces as a portal for the administration of drugs by topical application of the drug thereto for passage into the systemic circulation. As described herein, the stratum corneum can be disrupted in such transdermal drug transport.

“Biologically active agent” is to be construed in its broadest sense to mean any material that is intended to produce some biological, beneficial, therapeutic, or other intended effect, such as enhancing permeation or relief of pain. As used herein, the term “drug” refers to any material that is intended to produce some biological, beneficial, therapeutic, or other intended effect, such as relief of pain, but not agents (such as permeation enhancers) the primary effect of which is to aid in the delivery of another biologically active agent such as the therapeutic agent transdermally.

As used herein, the term “therapeutically effective” refers to the amount of drug or the rate of drug administration needed to produce the desired therapeutic result.

The terms “microprojections” and “microprotrusions”, as used herein, refer to piercing elements that are adapted to pierce or cut through the stratum corneum into the underlying epidermis layer, or epidermis and dermis layers, of the skin of a living animal, particularly a mammal and more particularly a human.

The term “microprojection array” or “microprotrusion array”, as used herein, refers to a plurality of microprojections arranged in an array for piercing the stratum corneum. The microprojection array may be formed by etching or punching a plurality of microprojections from a thin sheet and folding or bending the microprojections out of the plane of the sheet to form a configuration, such as the bent microproprojections shown in FIG. 2. Such methods of making microprojections are known in the art. For example, U.S. Pat. Nos. 5,879,326; 6,050,988; 6,091,975; 6,537,264 and US Patent Publication 20040094503 disclose processes for making microprojections by etching substrates. Silicon and plastic microprojection members are described in U.S. Pat. No. 5,879,326. The microprojection array can also be formed by other known methods, such as by forming one or more strips having microprojections along an edge of each of the strip(s) as disclosed in U.S. Pat. No. 6,050,988. These patent publications are incorporated herein by reference in their entireties.

The term “group” when referred to microprojection arrangement means a plurality, e.g., two (a pair), or more, of neighboring microprojections that are closer to one another than to other microprojections. In many cases, there are repeating units of such groups of microprojections in the microprojection array.

The present invention involves devices and methodology that provide increased drug loading per unit size of a microprojection member having a microprojection array for piercing the stratum corneum. Through sculpturing the microprojections to increase the surface area, a higher drug loading can be achieved compared to prior devices. For example, a microprojection can be sculptured to have a depression or cavity to increase surface area.

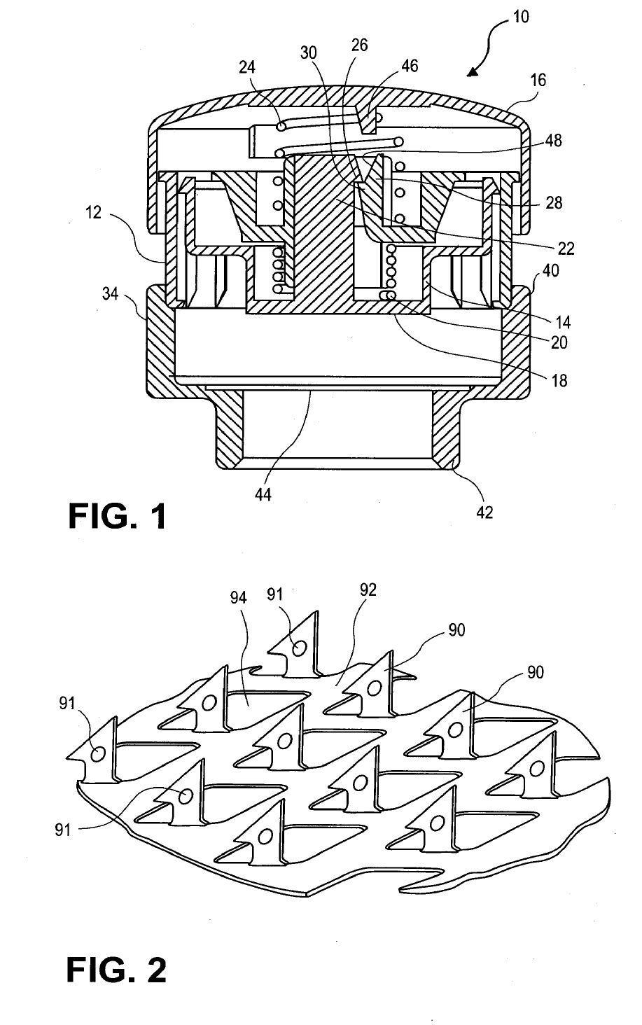

An applicator system for applying a microprojection member as described below includes an impact applicator for applying the microprojection member to the stratum corneum. The microprojection member can include a microprojection array. FIG. 1 shows a schematic sectional view of an exemplary microprojection device that can have a microprojection array of the present invention. Similar devices with actuators and microprojection members are described in United States patent documents 20020123675, 20050096586, 20050138926, 20050226922, and 20050089554, which are incorporated by reference herein. It is to be understood that such devices of these documents and other prior microprojection devices can be adapted to be used with the present invention. FIG. 1 illustrates an exemplary embodiment of an applicator 10 for use with the microprojection array of the present invention. However, the device of FIG. 1 is just an example and other applicator configurations may also be used with the microprojection arrays described herein. The applicator 10 includes a body 12 and a piston 14 movable within the body. A cap 16 is provided on the body 12 for activating the applicator to impact the stratum corneum with the microprojection member 44. An impact spring 20 is positioned around a post 22 of the piston 14 and biases the piston downward (i.e., towards the skin) with respect to the body 12. The piston 14 has an impact surface 18 that is substantially planar, slightly convex, or configured to match the contours of a particular body surface. The surface 18 of the piston 14 impacts the microprojection member 44 against the skin causing the microprojections 90 to pierce the stratum corneum of, for example, the skin of a patient.

FIG. 1 shows the piston 14 in a cocked position. When the applicator is cocked, the piston 14 is pressed up inside the body 12 and locked in place by a locking mechanism. The locking mechanism includes a stop catch 26 on the post 22 and a flexible finger 28 on the body 12 having a corresponding latch stop 30. As the piston 14 is moved toward the body 12 compressing the impact spring 20, the stop catch 26 flexes the finger 28 and snaps over the corresponding latch stop 30 of the flexible finger. The cocking step is performed by a single compression motion that both cocks and locks the piston 14 in the cocked position.

In the cocked position, catch 26 and latch 30 on the piston 14 and body 12 are releasably engaged, preventing downward motion of the piston in the body. FIG. 1 also illustrates the patch retainer 34 mounted on the body 12. The activation of the applicator 10 by the release of the locking mechanism is performed by downward force applied to the applicator cap 16 while the end 42 of the applicator is held against the skin. The cap 16 is biased in a direction away from the skin by a hold down spring 24 that is positioned between the body 12 and the cap. The cap 16 includes a pin 46 extending downward from the cap. When the cap 16 is pressed downward against the bias of the hold down spring 24, the pin 46 contacts ramp 48 on flexible finger 28 moving the flexible finger outward and disengaging latch 30 of the flexible finger 28 from catch 26. This releases piston 14 and the piston moves downward impacting the stratum corneum with the microprojection member 44. The impact is applied substantially parallel to a central axis of the microprojection member 44. Preferably, the microprojection member is connected to the retainer by at least one frangible element (not shown in the figure) that is broken when the impact applicator is activated.

FIG. 2 illustrates an exemplary embodiment of a microprojection member having a microprojection array of the present invention. FIG. 2 shows a plurality of microprojections (or microprotrusions) in the form of microblades or blade shaped microprojections 90, which have a blade shape with a cutting sharp point. The microblades or blade shaped microblades 90 extend at a substantially 90° angle from a sheet 92 having openings 94. The microprojections are preferably sized and shaped to penetrate the stratum corneum of the epidermis when pressure is applied to the microprojection member, for example, forming microslits on the body surface. The sheet 92 may be incorporated in an agent delivery patch or an agent-sampling patch that includes an agent (i.e., a pharmaceutical agent or drug) reservoir and/or an adhesive for attaching the patch to the stratum corneum.

Preferably the microprojections each have a drug coating with a drug (for example, on or near the tip of the microprojections). At least some of the microblades have a depression 91 on at least a face of the microblades. Such a depression will increase the surface area on which drug coating can adhere on the microblades 90 compared with microblades without the depression. Of course, some or all of the microblades in the microprojection member can have such a depression. Further, a single microblade can have multiple depressions and the depressions can have different shapes. The microprojection member and microprojection array can be made with technology known in the art. Examples of agent delivery and sampling patches that incorporate a microprojection array are found in US20020016562, U.S. Pat. No. 6,537,264, WO 97/48440, WO 97/48441, WO 97/48442, the disclosures of which are incorporated herein by reference in their entireties. The microprojection array of FIG. 2 without a drug reservoir or a drug coating may also be applied alone as a skin pretreatment. In one embodiment of the invention, the microprojections have projection length of less than 1000 microns (μ). In a further embodiment, the microprojections have a projection length of less than 500 microns (μ), more preferably, less than about 250μ. The microprojections preferably have a normally extending portion of about 25μ to 400μ long, more preferably about 50μ to 250μ long. As used herein, “normally extending” means extending at an angle from the plane of a microprojection member and, although possible, need not be exactly 90°.

The microprojections can be formed from metallic materials such as titanium, stainless steel, and polymers. Techniques for making microprojection array (e.g., by etching) from such materials are known in the art. Generally, substrates for forming microprojections are about 3 microns (μm) to 50 μm thick, preferably about 15 μm to 35 μm thick. The microprojections typically have a width of about 5 μm to 250 μm, preferably about 100 μm to 150 μm. The thicknesses of the microprojections are about 3 μm to 50 m, preferably about 10 μm to 30 μm. The microprojections may be formed in different shapes, such as needles, blades, pins, punches, and combinations thereof. If the microprojections are from the same sheet of material (for example, all were chemically etched from the same single sheet of titanium), the microprojection density is at least approximately 10 microprojections/cm², more preferably, in the range of approximately 200-5000 microprojections/cm². The distance between neighboring microprojections in a group can be about less than about 500 μm, preferably less than about 200 μm, even more preferably about 10 μm to 160 μm, even more preferably about 50 μm to 100 μm, at the base of the microprojections. Typically the microprojections extend from a base plate upward. The distance are generally measured between the base positions of the upwardly extending portions. There can be openings near the microprojections on the microprojection member. Such openings can allow agents or drugs to pass if agents or drugs are placed under or in such openings. The number of openings per unit area through which the active agent (drug) passes is preferably from approximately 10 openings/cm² to about 2000 openings/cm².

The depressions on the microprojections are small. Although various sizes are possible, generally the depressions are less than about 50 μm deep, preferably less than about 30 μm deep and less than about 50 μm wide, preferably less than about 30 μm wide, as they must be less wide than the microprojections and no deeper than the thickness of the microprojection, they are preferably formed by chemical etching.

Preferably the microprojections are blade-shaped to provide more surface area on the relatively flat surface and allow the sculpturing of the surface to form depressions. Further, a piece of material in sheet form lends itself to forming blade-shaped microprojections more readily than microprojections of other shapes.

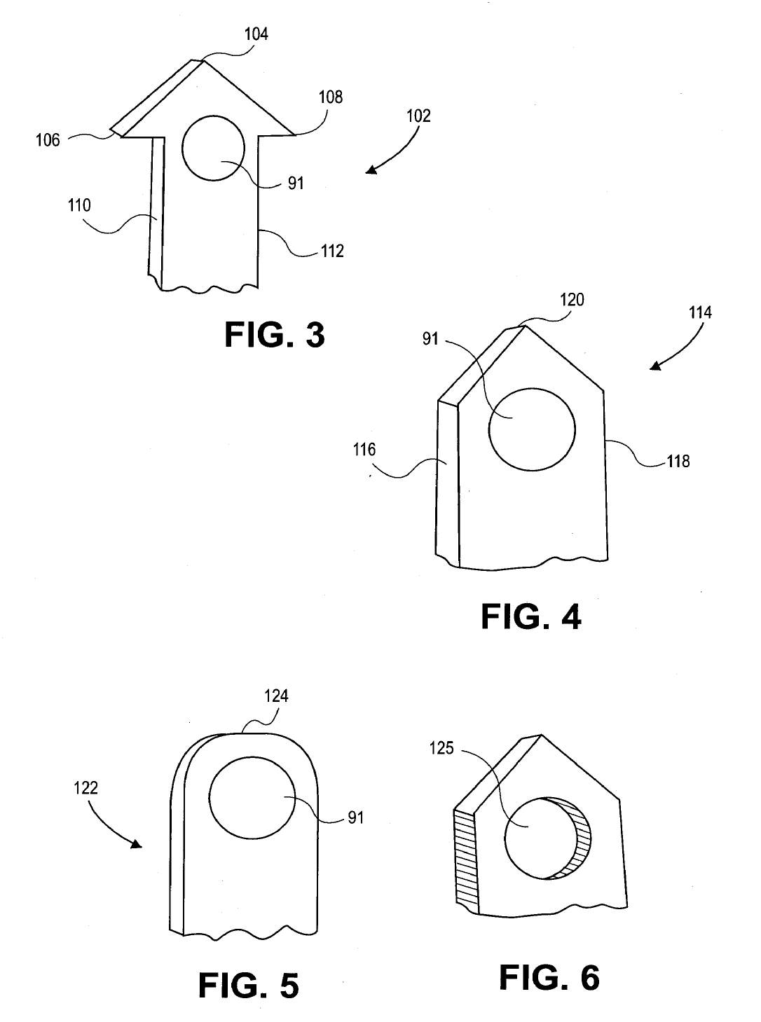

A microprojection can be sculptured (e.g., by chemical etching) to have different shapes and/or to form one or more depressions. For example, the microprojections of FIG. 2 have a top portion in half-arrowhead shape in that it has a shape point at the tip and one side edge but not on the other side edge. Another exemplary shape (shown in FIG. 3) for the top portion of a microprojection is arrowhead shaped, in which the microblade 102 is relatively flat and has a sharp pointed tip 104 on top. Two laterally extending protrusions with sharp points 106, 108 are located one on each side edge 110, 112 of the microblade. The microblade 102 is called a microblade because it is generally elongated and flat, although the edges 110 112 can be, but are not necessarily, sharp cutting edges for cutting into the body tissue of an individual. The cutting is done primarily by the tip 104 and its top (or distally) facing edge(s). “Distally” means the direction towards the skin surface when the device is to be applied. The arrowhead shaped microblade 102 also has a depression 91 on the face of the microblade. The microblade 102 thus has a “scoop” appearance, considering that it has a shaft and depression on its face. Further, in another embodiment, the pointed protrusions on the side edges of the microprojection can be rounded, thus forming a spade shape (not shown in the figures).

FIG. 4 shows yet another exemplary microprojection shape. Here, the microprojection, thus microblade 114, is tombstone shaped in that it does not have laterally extending lobes or sharp points on the side edges 116, 118. In this embodiment, the side edges 116, 118 are generally straight along the top portion of the microprojections and thus do not have the laterally extending points as those present in a barb or an arrowhead. A wedge shaped or pointed tip 120 is present at the end of the microblade 114. In the exemplary embodiment shown in FIG. 5, the tombstone shaped microblade 122 has a more rounded tip 124 than the embodiment shown in FIG. 3. Of course, a depression can be present on one or both faces, in a microblade design with arrowhead shape, or other shapes of this invention.

Further, as shown in exemplary FIG. 6, the depression on a microprojection can extend through the microblade forming a throughhole 125. In such a case, the depression can be considered to have joined with the depression on the other face of the microblade.

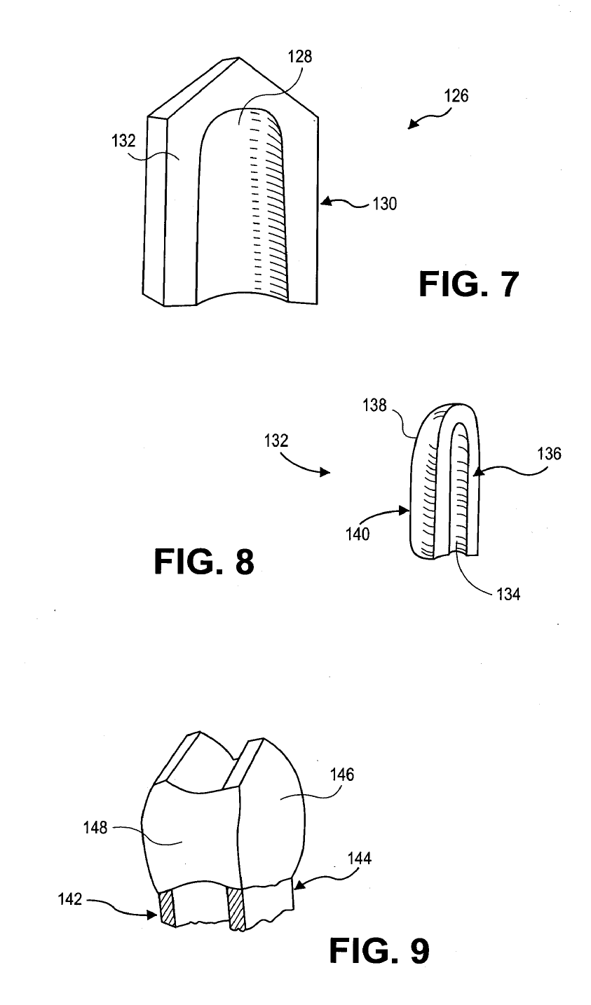

The depression that is on a microprojection can be generally round or oval in its outer perimeter, such as those shown in FIG. 3 to FIG. 6, or it can have other shapes such as star shaped, polygonal, etc. However, as exemplarily shown in FIG. 7, the microprojection, and thus microblade 126, can also have a depression 128 that is an elongated channel traversing along the elongated body 130 (or shaft) of the microblade 126 on its face 132. Further, the elongated channels can be connected on both sides to form elongated throughholes similar to shorter or more rounded depressions as described above. Of course, the microprojection with such elongated channel depressions, like those with a shorter, more rounded or oval depression, can have a wide variety of shapes, such as any of those described herein, e.g., arrowhead, tombstone, half arrowhead, and so on.

A further way to sculpture a microprojection is to not only sculpture one face of a microblade, but to sculpture both faces. One way to increase surface area, as mentioned before, is to have depressions on both faces. Further, in another alternative, as shown in FIG. 8, one face of a microblade can be sculptured to have a depression, such as a channel, and the face can have a more rounded, or bowed surface akin to a portion of an annular convex surface. For example, in FIG. 8, the microblade 132 has an elongated channel 134 on one face 136 and a bowed elongated back 138 on the opposite face 140. In this way, the microblade 132 has a top portion that is generally thumbnail shaped. The microblade 132 has the appearance of a scoop with a long trough on one side and the appearance of a curved sheet on the other side. Of course, the thumbnail appearance can have straight side edges as those in a tombstone design or have laterally extending points as in an arrowhead design.

A way to increase drug loading is to increase the amount of drug coating on a microprojection, as already mentioned. A further way to increase drug loading is to group neighboring microprojections close enough together to capture a continuous drug coating between the microprojections in the group. Thus, having a depression on at least one of the microprojections in the group will increase the volume of drug coating that can be held than otherwise without the depression. FIG. 9 illustrates an embodiment of a group (which in this case is a pair) of microprojections 142, 144 both of which have an elongated channel (not shown because it is covered by coating) on the face facing the other microprojection in the group. The microprojections 142, 144 extend in an about parallel fashion. A continuous drug coating 146 coats and extends from one microprojection 142 near its top to the other microprojection 144, forming a drug coating bridge 148. Thus, drug coating material bridges the microprojection 142, 144 and is sandwiched therebetween. Having the elongated channels on the microprojections thus increases the effective amount of drug coating that can be held by the two microprojections in the group. In another embodiment, one or more of the microprojections can have a channel facing away from the other microprojection.

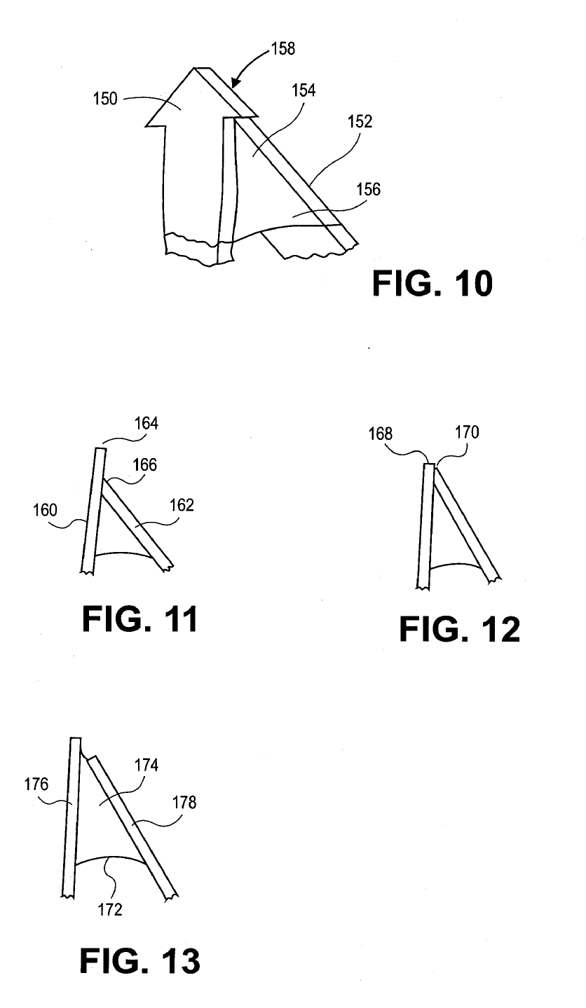

FIG. 10 shows an illustration of another alternative with a group (here a pair) of microprojections converging at the tips. In the embodiment of FIG. 10, microprojection 150 extends substantially straight up from the microprojection member planar plate (not shown) and microprojection 152 leans at an angle toward microprojection 150 so that the drug coating 154 forms a continuous bridge 156 coating the top portions of both of the microprojections. In this embodiment, microprojection 152 has an arrowhead shaped top portion and microprojection 152 has a tombstone shaped top portion. Both microprojections have a channel (not shown in the figure as being hidden behind the drug coating bridge 156) facing the other microprojection. The converging of microblades forms a pinnacle 158 that can facilitate penetration of the stratum corneum. The angle of leaning (relative to the plane of the microprojection member) preferably is about 60° to slightly less than 90°, more preferably about 70° to 80°. The leaning microprojection can be longer, the same length or shorter than the one that is not leaning.

The microblades can converge such that their tips are close together but not exactly touching. Alternatively, the microblades can converge to touch at the tips. Further, as shown in FIG. 11, one microblade (say, a first microblade) 160 can intercept a second microblade 162 along by the elongated portion of the first microblade 160 such that tip 164 of the first microblade 160 extends past the tip 166 and the body of the second microblade 162 (but not the other way around). The tip 166 of the second microprojection 162, although touching the first microprojection 160 in this embodiment, does not extend past the first microprojection. This way, during penetration of the stratum corneum, the tip 164 of first microblade 160 will initiate the penetration. Alternatively, the microblades can converge such that their tips 168, 170 are about even, as shown in FIG. 12. In this way, the tips 168, 170 of the microblades generally penetrate the stratum corneum at about the same time.

The proximity of microprojections in a group allows the drug coating liquid before solidifying to be drawn and held by capillary action among the microprojections in a group. This is especially useful in embodiments with converging top portions because the capillary action tends to draw the liquid drug coating towards the tips of the microprojections, and therefore at a position suitable to delivery drug deeper into the skin. This phenomenon is especially evident in instances in which hydrophilic drug coating is coating hydrophilic microprojections, wherein there is a small contact angle for the liquid on a surface. Wetability of a liquid on a surface is related to the contact angle α formed by the liquid-solid and the liquid-gas interfaces. If θ is greater than 90° the liquid tends to form droplets on the surface, i.e., the liquid does not wet the surface well. If θ is less than 90° the liquid tends to spread out over the surface. When the liquid forms a thin film on the surface i.e., wetting it well, θ tends to near zero. In instances of hydrophilic liquid on a hydrophilic surface, for example, as shown in FIG. 13, a concave shaped meniscus 172 would be formed by the capillary force in the drug coating 174 on the top portion of microprojections 176, 178 in a group. As used herein, even after the drug coating has solidified, the concaved shaped curve 172 is stilled called a meniscus for the sake of consistency. In FIG. 13, the tips of the microprojections 176, 178 do not actually touch. However, the drug coating 172, due to its viscosity before solidifying, still envelops the top portions of the microprojections and forms a bridge of continuous drug coating material between them. The bulk of the drug coating material is held between the microprojections in this embodiment.

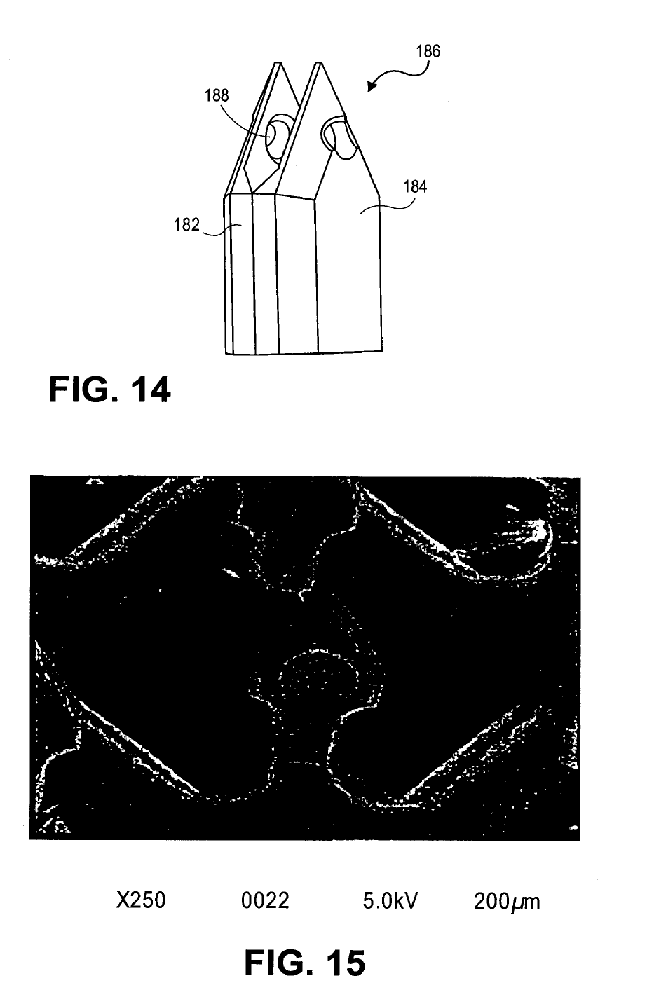

In yet another embodiment, as shown in FIG. 14, two microblades 182, 184 can pair up in close proximity (e.g., in contact) to form a composite microprojection 186. If preferred, throughholes 188 can be formed at the tips of the microblades 182, 184. Channels (troughs) can be forms on the face of each of the microblades to face the other microblade. When the two channels match in close proximity they form a tunnel in the composite microprojection 186. Drug (e.g., in a drug coating) can be put into the tunnel.

The convergence of the top portions of the microprojections in a group further functions to protect the drug coating from being pushed off the top portions of the microprojections because much of the drug coating is, for example, under the pinnacle formed by the tips of the microprojections and therefore shielded by the tips of the microprojections during penetration of the stratum corneum. In an embodiment in which the top portions of microprojections in a group are apart sufficiently on top at the tips as well as lower in the shafts of the microprojections, there can be a meniscus on the top of the drug coating as well as in the bottom of the drug coating.

A microprojection array can be made (or “sculptured”), for example, from a sheet of material by chemical etching. Methods for forming structures that are small (in the range of tens to hundreds of microns) by chemical etching are known in the art. A substrate material, generally flat as a sheet, such as a titanium sheet, can be chemically etched. In generally, a photoresist or a photo-sensitive polymer is laid on a substrate. A pattern is imaged on the photoresist (e.g., with ultra-violet light) and then the photoresist is then developed to provide a patterned polymer layer on the substrate. The patterned polymer layer protects portions of the substrate and leaves other portions unprotected. The substrate with the patterned polymer layer is exposed to an etching liquid, for example, as in a process of spraying the etching liquid on the substrate (with the patterned polymer layer thereon). The part of the substrate that is not protected by the patterned polymer layer is corroded, forming a patterned substrate having microblades that lie flat along the plane of the substrate. The microblades are then cleaned. The microblades are bent using dies. A microblade is bent such that an elongated portion extends normally from the plane of the substrate. This results in a microprojection array on a microprojection member.

In some embodiments, after a microprojection has been oriented, such as by lifting or bending a portion in the normal (i.e., generally perpendicular) direction, a portion of the microprojection extends along the plane of the substrate (the “planar portion”) to a bend. Past the bend, the normally extending portion projects upward from the plane of the substrate with the other microprojections, preferably in a regular pattern of repeated units of microprojections, to form the microprojection array. In certain designs, the planar portions in a group (e.g., a pair) of microprojections extend outward from one another (in a radiating form), although the top (distal) portions of the microprojections may converge. Such a design can be achieved, for example, by forming the microprojections in the group about a common spot of substrate material. In other designs, the planar portions of a group of microprojections extend toward one another (as opposite from a radiating form). Such a design can be achieved, for example, by stacking two layers of microprojections together so the microprojections of one layer protrude through openings of the other layer in a manner that in a group of microprojections the planar portion (extending along the plane of the base layer) of microprojection from one layer points toward the planar portion of microprojection of the other layer. Of course, yet another alternative is to have the two layers stacked together such that in a group one planar portion of microprojection of a first layer points toward a planar portion of microprojection of a second layer while the microprojection planar portion from the second layer points away from the microprojection planar portion of the first layer.

The drug coating can include one or more of a variety of drugs or biologically active agents. Such drugs or biologically active agents include traditional pharmaceuticals, as well as small molecules and biologics. Examples of such drugs or biologically active agents include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-i antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

The drugs or biologically active agents can also be in various forms, such as free bases, acids, charged or uncharged molecules, components of molecular complexes or nonirritating, pharmacologically acceptable salts. Further, simple derivatives of the active agents (such as ethers, esters, amides, etc.), which are easily hydrolyzed at body pH, enzymes, etc., can be employed.

The drugs or biologically active agents can be incorporated into a liquid drug coating material and coated onto the microprojections.

Typically, the drug or biologically active agent is present in the drug coating formulation at a concentration in the range of approximately 0.1-30 wt %, preferably 1-30 wt %.

Preferably, the amount of drug contained in the biocompatible coating (i.e., dose) is in the range of approximately 1 μg-1000 μg, more preferably, in the range of approximately 10-200 μg per dosage unit. Even more preferably, the amount of the drug contained in the biocompatible coating is in the range of approximately 10-100 μg per dosage unit.

Preferably, the pH of the coating formulation is adjusted to provide conditions for maintaining the stability of the drug selected for incorporation in the drug coating formulation. In certain embodiments of the invention, the viscosity of the coating formulation is enhanced by adding low volatility counterions. In certain embodiments, the drug has a positive charge at the formulation pH and the viscosity-enhancing counterion comprises an acid having at least two acidic pKas. Suitable acids include, without limitation, maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid and phosphoric acid.

In some of the embodiments of the invention, the amount of counterion is preferably sufficient to neutralize the charge of the drug. In such embodiments, the counterion or the mixture of counterion is preferably sufficient to neutralize the charge present on the agent at the pH of the formulation. In additional embodiments, excess counterion (as the free acid or as a salt) is added to the drug to control pH and provide adequate buffering capacity.

In one embodiment, the counterion comprises a viscosity-enhancing mixture of counterions chosen from the group consisting of citric acid, tartaric acid, malic acid, hydrochloric acid, glycolic acid and acetic acid. Preferably, the counterions are added to the formulation to achieve desired viscosity.

The viscosity of the drug coating formulation in liquid form is affected by the nature of the polymeric material and counterions present. The drug coating formulations have a viscosity of less than approximately 500 centipoise (typically measured at 25° C. and at a shear strain rate of 100/sec) and greater than 3 centipoise (cp), preferably a viscosity in the range of about 20-200 cp. Such viscosity ranges are suitable for forming a drug coating on the microprojections, for example, wherein capillary force can hold the liquid drug coating formation between the microprojections in a group until the formulation is solidified.

In certain embodiments, the viscosity-enhancing counterion contains an acidic counterion, such as a low volatility weak acid. Preferably, the low volatility weak acid counterion exhibits at least one acidic pKa and a melting point higher than about 50° C. or a boiling point higher than about 170° C. at atmospheric pressure. Examples of such acids include, without limitation, citric acid, succinic acid, glycolic acid, gluconic acid, glucuronic acid, lactic acid, malic acid, pyruvic acid, tartaric acid, tartronic acid and fumaric acid.

In another embodiment, the counterion comprises a strong acid. Preferably, the strong acid exhibits at least one pKa lower than about 2. Examples of such acids include, without limitation, hydrochloric acid, hydrobromic acid, nitric acid, sulfonic acid, sulfuric acid, maleic acid, phosphoric acid, benzene sulfonic acid and methane sulfonic acid. Another embodiment is directed to a mixture of counterions, wherein at least one of the counterion comprises a strong acid and at least one of the counterions comprises a low volatility weak acid.

Another preferred embodiment is directed to a mixture of counterions, wherein at least one of the counterions comprises a strong acid and at least one of the counterions comprises a weak acid with high volatility. Preferably, the volatile weak acid counterion exhibits at least one pKa higher than about 2 and a melting point lower than about 50° C. or a boiling point lower than about 170° C. at atmospheric pressure. Examples of such acids include, without limitation, acetic acid, propionic acid, pentanoic acid and the like.

The acidic counterion is preferably present in an amount sufficient to neutralize the positive charge present on the drug at the pH of the formulation. In additional embodiments, excess counterion (as the free acid or as a salt) is added to control pH and to provide adequate buffering capacity.

In another embodiment of the invention, the coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

In one embodiment of the invention, the coating formulation includes at least one antioxidant, which can be sequestering agents, such sodium citrate, citric acid, EDTA (ethylene-dinitrilo-tetraacetic acid) or free radical scavengers such as ascorbic acid, methionine, sodium ascorbate and the like. Presently preferred antioxidants comprise EDTA and methionine.

In the noted embodiments of the invention, the concentration of the antioxidant is in the range of approximately 0.01-20 wt. % of the coating formulation. Preferably the antioxidant is in the range of approximately 0.03-10 wt. % of the coating formulation.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, alkoxylated alcohols, such as laureth-4 and polyoxyethylene castor oil derivatives, such as CREMOPHOR EL.

In one embodiment of the invention, the concentration of the surfactant is in the range of approximately 0.01-20 wt % of the coating formulation. Preferably the surfactant is in the range of approximately 0.05-1 wt % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcell-ulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.01-20 wt %, more preferably, in the range of approximately 0.03-10 wt. % of the coating formulation.

In another embodiment, the coating formulation includes a hydrophilic polymer selected from the following group: hydroxyethyl starch, carboxymethyl cellulose and salts of, dextran, poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 1-30 wt %, more preferably, in the range of approximately 1-20 wt % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose, stachyose, mannitol, and other sugar alcohols.

Preferably, the concentration of the biocompatible carrier in the coating formulation is in the range of approximately 2-70 wt %, more preferably, in the range of approximately 5-50 wt % of the coating formulation.

In another embodiment, the coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and insulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

Preferably, the concentration of the stabilizing agent in the coating formulation is at ratio of approximately 0.1-2.0:1 with respect to the drug, more preferably, approximately 0.25-1.0:1 with respect to the drug.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tyrnazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline. The concentration of the vasoconstrictor, if employed, is preferably in the range of approximately 0.1 wt % to 10 wt % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin. Most preferred solubilising/complexing agents are beta-Cyclodextrin, hydroxypropyl beta-Cyclodextrin, 2-hydroxypropyl-beta-Cyclodextrin and sulfobutylether7 beta-Cyclodextrin. The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt % to 50 wt % of the coating formulation. Other known formulation adjuvants can also be added to the coating formulations provided they do not adversely affect the necessary solubility and viscosity characteristics of the coating formulation and the physical integrity of the dried coating.

In one embodiment of the invention, the thickness of the dry biocompatible coating (drug coating) is less than 25μ, more preferably, less than 10μ, as measured from the microprojection surface. The desired coating thickness is dependent upon several factors, including the required dosage and, hence, coating thickness necessary to deliver the dosage, the density of the microprojections per unit area of the sheet, the viscosity and concentration of the coating composition and the coating method chosen.

In accordance with one embodiment of the invention, the method for delivering a drug contained in the biocompatible coating on the microprojection member includes the following steps: the coated microprojection member is initially applied to the patient's skin via an actuator, wherein the microprojections pierce the stratum corneum. The coated microprojection member is preferably left on the skin for a period lasting from 5 seconds to 24 hours. Following the desired wearing time, the microprojection member is removed.

The drug coating can be formed on microprojections by using rollers, for example, with the method and apparatus described by U.S. patent publication 20020132054, which in incorporated by reference herein in its entirety. Briefly described, a coating liquid containing a drug is conveyed to a liquid holding surface having a coating transfer region, such as a surface of a rotating drum. A microprojection member having a microprojection array is passed over the coating transfer region such that the microprojections dip their top portions into the coating liquid at the desired depth. The depth of the coating liquid at the coating transfer region is controlled so that right amount of drug coating liquid is deposited on the microprojection at the right height on the microprojection. The depth of the coating liquid at the coating transfer region can be controlled, for example, by using a doctor blade.

After a liquid drug coating has been deposited on the microprojections, the liquid drug coating is dried to solidify the liquid drug coating. The drying can be done at ambient (room) conditions. Further, various drying techniques can be used, such as using heat, controlled lower vapor pressure of the solvent in atmosphere above the liquid, etc.

The microprojection array can be applied on the skin of an individual, for example, by using an applicator, as done by other conventional microprojection arrays.

### EXAMPLES

Below are examples of specific embodiments for carrying out the present invention. The examples are offered for illustrative purposes only, and are not intended to limit the scope of the present invention in any way.

### Example 1

A first substrate titanium sheet about 50μ thick is coated with photoresist, imaged for a pattern to form microblades and chemically etched with etching solutions, such as ferric chloride solution, known in the art. The patterned polymer layer protects portions of the substrate and leaves other portions unprotected. After ectching, the part of the substrate that is not protected by the patterned polymer layer is corroded, forming a patterned substrate having microblades that lie flat along the plane of the substrate. The microblades are then cleaned and bent using dies. The microblades are etched to have a channel on one side of the microblades. Each microblade is bent such that an elongated portion extends normally from the plane of the substrate about 150μ long and 50μ wide. A first microblade array with openings similar to FIG. 2 is formed.

A second substrate titanium sheet is similarly photoresist coated, imaged and etched as described above. The microblades are etched to have a channel on one side of the microblades and each microblade is bent such that an elongated portion extends normally from the plane of the substrate. A channel is etched into a side of the microblade from the second substrate sheet to face a corresponding channel of the microblade from the first substrate sheet (considering when the two microblade arrays are stacked together). A second microblade array with openings similar to FIG. 2 is formed.

A microprojection array is formed by stacking the first microblade array with the second microblade array so that the microblades of one array protrude through the openings in the other array so that microblades of the two array contact and match with the channels facing each other. FIG. 14 shows a microprojection formed from a microblade from the first substrate sheet matching with a microblade from the second substrate sheet. By stacking the first microblade array with the second microblade array, a microblade 182 from the first substrate sheet when placed next to and contacting a matching microblade 184 from the second substrate sheet to form a composite microprojection 186. The two channels of the two adjoining corresponding microproblades match to form a tunnel (not shown because it is hidden from view) in the composite microprojection 186. This tunnel is a void or cavity that is then filled with drug in the form of a drug coating. A drug coating known in the art can be used, e.g., those disclosed in US Patent Publications 20020132054, 20050256045. (For example, US Patent Publication 20020132054 discloses drug coatings with human growth hormone and US Patent Publication 20050256045 discloses drug coatings with parathyroid hormone.) A throughhole 188 can also be formed near the tip of each microblade. This results in a microprojection array on a microprojection member. When the composite microprojection penetrates the skin the drug dissolves in the interstitial fluid and is drawn into the skin by diffusion.

### Example 2

A first substrate titanium sheet was etched in a process similar to that described in Example 1 to form a first microblade array. The normally projecting microblades were about 225μ length, 116μ width, 25μ thickness, and having an arrowhead. A depression was formed in each microblade in the chemical etching. The depression was approximately 65μ wide by 90μ tall and 15μ deep.

A second substrate titanium sheet was etched in a process similar to that described in Example 1 to form a second microblade array. However, the depression was formed on each of the microblades on a face that faced away from the corresponding matching microblade from the first microblade array when the first and the second microblade array were stacked together. FIG. 15 is a scanning electronmigraph showing a portion of the microprojection array that resulted from stacking the first microblade array with the second microblade array such that the microblades from one array protruded through the openings of the other array (as shown in the electronmicrograph). The microblade from the first microblade array was spaced about 200μ from the corresponding matching microblade from the second microblade array. This formed a composite microprojection array. Drug coating can be coated on the microprojection array with any coating process known in the art, e.g., using a coating machine similar to that described in U.S. Patent Publication 20020132054.

### Example 3

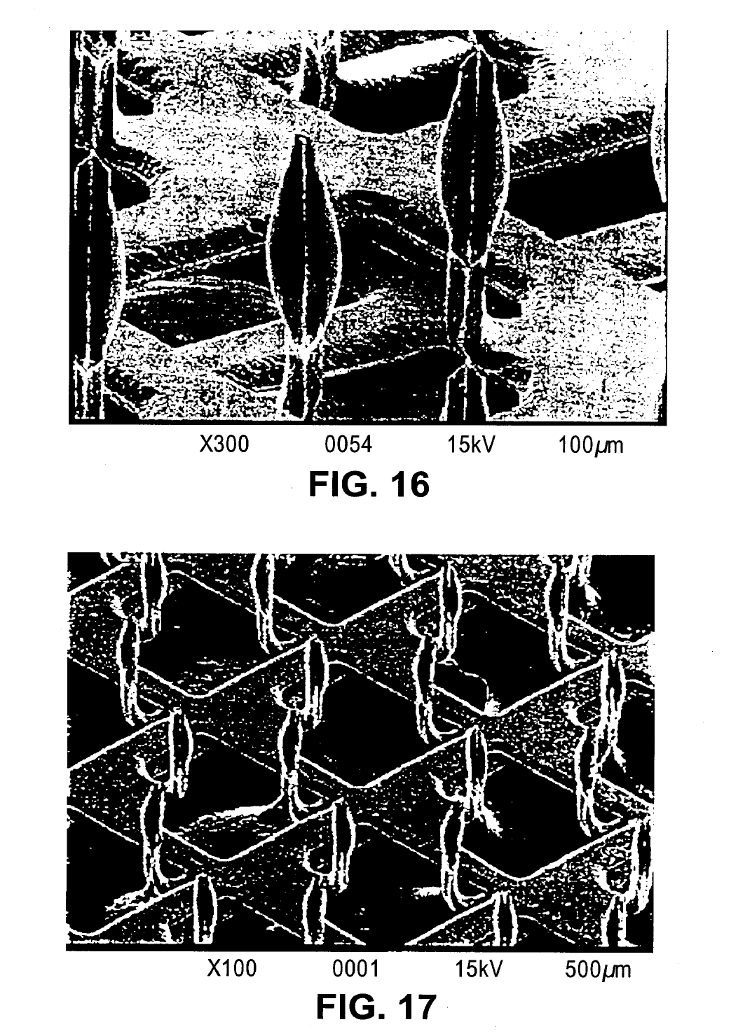

A microprojection array having microblades from two microblade arrays tacked together was formed by a process similar to that of Example 2, except that no depression was formed on any of the microblades. The top portions of the microblades of the microprojection array were coated with a drug coating. When dried and the solvent evaporated, the drug coating solids remaining on the microblades averaged out to be about 138 nanograms (ng) per microblade. FIG. 16 is a scanning electronmigraph showing a portion of the microprojection array that resulted from stacking the first microblade array with the second microblade array and coating the top portions of the microblades with a drug coating. Since both faces of a microblade were similarly without depression and were flat, the surfaces of the solid drug coating on both faces had similar profiles and look symmetrical from a side view.

### Example 4

A microprojection array having microblades from two microblade arrays tacked together was formed by a process similar to that of Example 2 to result in microblades in a pair facing each other but spaced apart as in Example 3, except that a depression was formed on each of the microblades similar to Example 2, unlike Example 3. However, except for the depressions, the microprojection array of microblades of Example 3 was the same as the microprojection array here in Example 4. The top portions of the microblades of the microprojection array were coated with a drug coating. When dried and the solvent evaporated, the drug coating solids remaining on the microblades averaged out to be about 141 nanograms (ng) per microblade. This showed that such a depression on a microblade increased its copacity to hold drugs compared to a similar microblade without a depression. FIG. 17 is a scanning electronmigraph showing a portion of the microprojection array that resulted from stacking the first microblade array with the second microblade and coating the top portions of the microblades with a drug coating. Due to the presence of a depression on a face, the face with the depression tended to have a flatter drug coating surface and three dimensional profile than the face without the depression. Thus, from a side view, the two sides (faces) of the drug coating are asymmetrical.

The entire disclosure of each patent, patent application, and publication cited or described in this document is hereby incorporated herein by reference. The practice of the present invention will employ, unless otherwise indicated, conventional methods used by those in pharmaceutical product development within those of skill of the art. Embodiments of the present invention have been described with specificity. The embodiments are intended to be illustrative in all respects, rather than restrictive, of the present invention. It is to be understood that various combinations and permutations of various constituents, parts and components of the schemes disclosed herein can be implemented by one skilled in the art without departing from the scope of the present invention.

What is claimed is:

1. An apparatus for stratum-corneum piercing drug delivery, comprising: microprojection array having a plurality of stratum-corneum piercing microprojections for piercing stratum-corneum to facilitate drug delivery wherein at least some of the microprojections are microprojections having a surface with a depression thereon, and a drug coating disposed on at least a portion of the depression.

2. The apparatus of claim 1, wherein at least some of the microprojections having depressions are blade shaped microprojections.

3. The apparatus of claim 2, wherein the blade shaped microprojection has a sharp cutting point.

4. The apparatus of claim 2, wherein the depression is located on one side of the microprojection.

5. The apparatus of claim 2, wherein the depression is located on at least one side of the microprojection.

6. The apparatus of claim 2, wherein at least some of the microprojections have depressions on two side of the microprojection.

7. The apparatus of claim 2 wherein the microprojections have shafts and at least some of the depressions are elongated along at least a portion of the shafts.

8. The apparatus of claim 2 wherein the microprojections have shafts and at least some of the depressions are elongated along their respective shafts and at least some of the microprojections have a curved surface bowing oppositely from the depression.

9. The apparatus of claim 2 wherein at least some of the microprojections have depressions on two sides of a blade shaped microprojection forming a throughhole.

10. The apparatus of claim 2 wherein at least some the microprojections have an arrowhead tip or a tombstone tip.

11. The apparatus of claim 2 wherein at least some of the microprojections have an arrowhead tip or a tombstone tip and some microprojection are without either an arrowhead or a tombstone tip.

12. An apparatus for stratum-corneum piercing drug delivery, comprising: a microprojection array having a plurality of stratum-corneum piercing microprojections for piercing stratum-corneum to facilitate drug delivery, at least some of the microprojections having a surface with an elongated channel depression thereon, a drug coating on at least a portion of the microprojection covering the elongated channel depression.

13. An apparatus for stratum-corneum piercing drug delivery, comprising: a microprojection array having a plurality of stratum-corneum piercing microprojections for piercing stratum-corneum to facilitate drug delivery, at least some of the microprojections are thumbnail shaped having a surface with an elongated channel depression thereon, a drug coating disposed on at least a portion of the elongated channel depression of the microprojection.

14. An apparatus for stratum-corneum piercing drug delivery, comprising: a microprojection array having a plurality of stratum-corneum piercing microprojections for piercing stratum-corneum to facilitate drug delivery, at least some of the microprojections having a surface with a depression thereon, a drug coating on at least a portion of the microprojection disposed on the depression, at least some of the microprojections forming groups.

15. The apparatus of claim 14 wherein at least some of the microprojections are together in pairs and in the pair at least one microprojection projects at an angle to lean toward the other microprojection in the pair.

16. The apparatus of claim 14 wherein at least some of the microprojections are together in pairs and in a pair the microprojections have top portions that are substantially parallel.

17. The apparatus of claim 14 wherein each microprojection has a base.

18. The apparatus of claim 17 wherein at least some of the microprojections are together in pairs and wherein the bases of the pair of microprojections are spaced apart at the base by less than 200 μm.

19. The apparatus of claim 17 wherein at least some of the microprojections are together in pairs and wherein the bases of the pair of microprojections are spaced apart at the bases by 10 μm to 100 μm.

20. The apparatus of claim 14 wherein at least some of the microprojections are together in pairs and a drug coating coats a pair as a continuous coating.

21. The apparatus of claim 14 wherein each microprojection has a tip and wherein at least some of the microprojections are together in pairs and a drug coating coats the pair as a continuous coating near the tips.

22. The apparatus of claim 14 wherein at least some of the microprojections are together in pairs and in the pair each microprojections of the pair includes a depression and a drug coating coats the pair as a continuous coating.

23. The apparatus of claim 14 wherein at least some of the microprojections are together in pairs and in the pair only one microprojection of the pair has a depression and a drug coating coats the pair as a continuous coating.

24. The apparatus of claim 14 wherein at least some of the microprojections are together in pairs and in the pair at least one microprojection in the pair has a depression facing the other microprojection of the pair and a drug coating coats the pair as a continuous coating.

25. A method for stratum-corneum piercing drug delivery to an individual, comprising: providing (a) a plurality of stratum corneum piercing microprojections for piercing stratum corneum to facilitate drug delivery, (b) providing at least some of the microprojections to have a surface with a depression thereon, (c) coating a drug on at least a portion of the microprojection covering the depression, and (d) piercing the stratum corneum of said individual with the microprojections.

26. The method of claim 25 providing blade shaped microprojections having depressions on one side of the blade shaped microprojection.

27. The method of claim 25 providing microprojections having shafts and at least some of the depressions are elongated along at least portion of their respective shafts.

28. The method of claim 25 providing at least some of the microprojections having a throughhole.

29. The method of claim 25 providing at least some of the blade shaped microprojections having depressions on two sides of a blade.

30. A method for forming a stratum-corneum piercing drug delivery apparatus, comprising: (a) forming a plurality of stratum-corneum piercing microprojections for piercing the stratum-corneum to facilitate drug delivery, (b) forming a depression on the surface of at least some of the microprojections, and (c) coating a drug on at least a portion of the microprojection depression.

31. The method of claim 30 further comprising forming blade shaped microprojections having a depression on one side of a blade.

32. The method of claim 30 comprising forming on at least some of the microprojections shafts and further forming on at least some of the microprojections depressions as elongated channels along at least a portion of their respective shafts.

33. A method for forming a stratum-corneum piercing drug delivery apparatus, comprising: (a) forming a plurality of stratum-corneum piercing microprojections for piercing stratum-corneum to facilitate drug delivery, (b) forming a depression on the surface of at least some of the microprojections, (c) coating a drug on at least a portion of the microprojection depression, and (d) positioning the microprojections in groups.

34. The method of claim 33 comprising forming at least some of the microprojections to associate in pairs and wherein at least one of the microprojections in the pair projects at an angle to lean toward the other microprojection in the pair.

35. The method of claim 33 comprising forming at least some of the microprojections to associate in pairs and wherein at least one pair of microprojections has a continuous drug coating.

36. The method of claim 33 comprising a plurality of microprojections each microprojection having a base and wherein at least some of the microprojections associate in pairs, wherein the bases of the microprojections in the pair are set apart by less than 200 μm.
